(19) United States
(12) Patent Application Publication
(10) Pub. No.: US 2024/0342137 A1
(43) Pub. Date: Oct. 17, 2024

(54) COMPOSITIONS OF ALPHA-2 ANTAGONISTS AND METHODS AND USES THEREOF

(71) Applicant: Rodney J.Y. Ho, Mercer Island, WA (US)

(72) Inventor: Rodney J.Y. Ho, Mercer Island, WA (US)

(21) Appl. No.: 18/628,666

(22) Filed: Apr. 5, 2024

Related U.S. Application Data
(60) Provisional application No. 63/495,549, filed on Apr. 11, 2023.

(51) Int. Cl. A61K 31/4172; A61K 31/4168; A61K 31/4174; A61K 31/54

(52) U.S. Cl. CPC A61K 31/4172; A61K 31/4168; A61K 31/4174; A61K 31/54

(57) ABSTRACT

An embodiment includes a composition for reversing an alpha-2 adrenoceptor agonist in a subject comprising an alpha-2 antagonist and a histidine in a pharmaceutically acceptable formulation. The embodiment includes where the composition comprises a pi-pi interaction between the alpha-2 antagonist and the histidine. The embodiment also includes where the alpha-2 antagonist comprises from about 80 mg/mL to 97.6 mg/mL.

### CLAIM OF PRIORITY

This application claims the benefit of U.S. Provisional Patent Application No. 63/495,549 filed on Apr. 11, 2023.

### BACKGROUND

The present invention relates generally to alpha-2 antagonists. More particularly, the present invention relates to Compositions of Alpha-2 Antagonists and Methods and Uses Thereof.

Alpha-2 antagonists bind to and block the activation of adrenergic alpha-2 agonists such as xylazine, medetomidine, dexmedetomidine, brimonidine, and clonidine. Examples of alpha-2 antagonists include atipamezole, marketed as veterinary products Antisedan® and Revertidine® that are currently approved only for veterinary use. Current atipamezole products are not suitable or approved for human application. Dexmedetomidine is an alpha agonist having sedative, anxiolytic, hypnotic, analgesic, and sympatholytic properties. Dexmedetomidine is commonly used in anesthesia practice. The adrenergic alpha-2 agonist Xylazine is a non-opioid veterinary tranquilizer or sedative that is not approved for human use. Xylazine is increasingly found in illicit drug and opioid supply in a majority 48 of 50 states in the United States and is linked to increased overdose deaths across the country. Moreover, some fentanyl street products are also laced with xylazine where fentanyl in combination with xylazine is often used to extend the effects of fentanyl and mimic the high of heroin. With the opioid epidemic and the wide availability of opioids such as fentanyl and other opioid street drugs, there are growing opioid overdose cases across the United States.

### SUMMARY

The illustrative embodiments provide for Compositions of Alpha-2 Antagonists and Methods and Uses Thereof. An embodiment includes a composition for reversing an alpha-2 adrenoceptor agonist in a subject comprising an alpha-2 antagonist and a histidine in a pharmaceutically acceptable formulation.

### BRIEF DESCRIPTION OF THE DRAWINGS

The novel features believed characteristic of the invention are set forth in the appended claims. The invention itself, however, as well as a preferred mode of use, further objectives, and advantages thereof, will best be understood by reference to the following detailed description of the illustrative embodiments when read in conjunction with the accompanying drawings, wherein:

FIG. 1A depicts atipamezole in accordance with an illustrative embodiment;

FIG. 1B depicts an atipamezole hydrogen chloride (HCl) structure in accordance with an illustrative embodiment;

FIG. 2A depicts histidine in accordance with an illustrative embodiment;

FIG. 2B depicts an atipamezole-histidine complex in accordance with an illustrative embodiment; and

FIG. 3 depicts a chart showing the effects of dose-response on histidine to dissolve about 800 mg or 3.768 millimole (mmol) of atipamezole in 10 mL suspension in accordance with an illustrative embodiment.

### DETAILED DESCRIPTION

Alpha-2 antagonists bind to alpha-2 adrenoreceptors and block the activation of adrenergic alpha-2 agonists such as xylazine, medetomidine, dexmedetomidine, brimonidine, and clonidine. Examples of alpha-2 antagonists include atipamezole, branded as Antisedan® and Revertidine® that are currently approved only for veterinary use. Current veterinary atipamezole products are not suitable or approved by the FDA for use in humans. Dexmedetomidine is an alpha-2 agonist having sedative, anxiolytic, hypnotic, analgesic, and sympatholytic properties. Dexmedetomidine is commonly used in anesthesia practice. The adrenergic alpha-2 agonist xylazine is a non-opioid veterinary tranquilizer or sedative that is not approved for human use. Xylazine is increasingly found in illicit drug and opioid supply in a majority 48 of 50 states in the United States and is linked to increased overdose deaths across the country. Moreover, some fentanyl street products are also laced with xylazine where fentanyl in combination with xylazine is often used to extend the effects of fentanyl and mimic the high of heroin. With the opioid epidemic and the wide availability of opioids such as fentanyl and other opioid street drugs, there are growing opioid overdose cases across the US.

Atipamezole is a potent and well-characterized antagonist of the alpha-2 adrenergic receptor. It is currently approved only for veterinary use (NADA-242-033) for reversing adrenergic alpha-2 agonists including but not limited to xylazine, medetomidine, dexmedetomidine, brimonidine, and clonidine. The brand names of atipamezole, Antisedan® and its generic form Revertidine® are currently marketed as a 5 mg/mL injectable veterinary product, but not approved for human use.

Xylazine, branded as Rompun®, is a non-opioid veterinary tranquilizer or sedative that is not approved for human use. According to the National Institute on Drug Abuse (NIDA), xylazine has been linked to an increasing number of illicit drug overdose deaths nationwide in the evolving drug addiction and opioid epidemic crises. (Reyes et al. 2012) (Friedman et al. 2022) (Thangada et al. 2021) (Johnson et al. 2021). Xylazine is not an opioid, and its drug effect is not mediated through an opioid receptor. Instead, xyalzine's sedative and anesthetic effect is mediated through adrenergic alpha-2 receptor. Consequently, opioid-overdose reversal agents such as naloxone and naltrexone that bind to opioid receptors are not effective (Gallanosa et al. 1981). Research has also shown that people using illicit opioids such as fentanyl are either knowingly or unknowingly taking fentanyl laced with xylazine to lengthen its euphoric effects, which may lead to severe respiratory and cardiovascular events and even death in the case of an overdose.

Xylazine, known also by its street names “zombie drug”, “tranq” and “tranq dope”, is a non-opioid veterinary tranquilizer or sedative. It is not approved for human use; it is approved only for veterinary use is dogs, horses, cattle and other mammals. Illicit use of Xylazine can be life threatening and is deadly when combined with opioids such as fentanyl.

The opioid epidemic, coupled with the wide availability of opioids such as fentanyl and other related drugs, has led to ever growing cases of opioid overdose across the US and across all ages. To save lives in opioid-related overdose situations, opioid reversal agents such as naloxone (intranasal dosage form as well as injectable dosage form) and naltrexone are now made available over the counter, through standing order to dispense [naloxone] to a “natural person” such as an individual at risk of an opioid-related overdose, a family member, friend or an acquaintance of that individual; or to a “legal person” such as an ambulance service, police, school or other educational institutions that could be in a position to assist a person at risk of experiencing an opioid-related overdose (Washington State Department of Health Naloxone Standing Order for Dispensing). As recently as April 2023, FDA approved an over-the-counter use of Narcan (naloxone) intranasal product and prescription is no longer necessary to gain access to this product. Across the United States, many such standing orders for opioid reversal agents like naloxone in ready-to-use injectable and nasal spray dosage forms have proven to save lives from opioid overdose.

However, because naloxone is a specific antagonist for an opioid receptor, it will not reverse the effects of xylazine in overdose cases caused by fentanyl laced with xylazine. As such, respiratory and cardiovascular events caused by xylazine in overdose situations may continue even after naloxone is administered. Xylazine is not opioid, but it is a well-established agent that mediates sedation mainly through binding to adrenergic alpha-2 receptor. Therefore, the overdose and untoward events caused by alpha-2 agonists such as xylazine can only be reversed with administration of alpha-2 antagonists that inhibit alpha-2 receptors, such as atipamezole.

Among the alpha-2 antagonists reported, atipamezole is one the most potent inhibitor with half-maximal inhibitory concentration at 1.6 nM, compared to other alpha-2 specific inhibitors such as idazoxan and yohimbine which are over 80 fold lower sensitivity (148 and 130 nM for this measure) (Pertovaara et al. 2006). Atipamezole is approved only for veterinary use for reversing alpha-2 agonist including xylazine.

The current veterinary injectable dosage form of alpha-2 antagonists may be considered for reversing xylazine overdose in subjects; however, the current formulations achieved in the veterinary product are not of sufficient concentration, or quality.

The present disclosure addresses the deficiencies described above by providing compositions, uses and methods for Compositions of Alpha-2 Antagonists and Methods and Uses Thereof. An embodiment includes a composition for reversing an alpha-2 adrenoceptor agonist in a subject comprising an alpha-2 antagonist and a histidine in a pharmaceutically acceptable formulation.

Illustrative embodiments include where the composition comprises a pi-pi interaction between the alpha-2 antagonist and the histidine.

Illustrative embodiments include where the alpha-2 antagonist comprises from about 80 mg/mL to 97.6 mg/mL.

Illustrative embodiments include where the histidine comprises from about 300 mM to 400 mM.

Illustrative embodiments include where the alpha-2 adrenoceptor agonist is selected from the group consisting of xylazine, dexmedetomidine and clonidine.

Illustrative embodiments include where the alpha-2 antagonist is atipamezole.

Illustrative embodiments include where the composition is administered via a route selected from the group consisting of intravenous administration, injection administration, nasal administration and buccal administration.

Illustrative embodiments also include where the composition has a pH of about 5.5 to about 6.

For the sake of clarity of the description, and without implying any limitation thereto, the illustrative embodiments are described using some example configurations. From this disclosure, those of ordinary skill in the art will be able to conceive many alterations, adaptations, and modifications of a described configuration for achieving a described purpose, and the same are contemplated within the scope of the illustrative embodiments.

Furthermore, the illustrative embodiments are described with respect to specific actual or hypothetical components only as examples. Any specific manifestations of these and other similar artifacts are not intended to be limiting to the invention. Any suitable manifestation of these and other similar artifacts can be selected within the scope of the illustrative embodiments.

The examples in this disclosure are used only for the clarity of the description and are not limiting to the illustrative embodiments. Any advantages listed herein are only examples and are not intended to be limiting to the illustrative embodiments. Additional or different advantages may be realized by specific illustrative embodiments. Furthermore, a particular illustrative embodiment may have some, all, or none of the advantages listed above.

The examples in this disclosure are used only for the clarity of the description and are not limiting to the illustrative embodiments. The formulations and methods disclosed in the embodiments herein may comprise or cover the essential steps, elements, and where appropriate limitations of the invention described here. In addition, optional ingredients, components, steps or process, or limitations are described to enable making a high concentration of pharmaceutically acceptable antidote product compositions.

The disclosed composition may comprise alpha-2 antagonists, such as atipamezole (4-(2-ethyl-2,3-dihydro-1H-inden-2-yl)-1H-imidazole), atipamezole-HCl (4-(2-ethyl-2,3-dihydro-1H-inden-2-yl)-1 H-imidazole hydrochloride), 5-(2-ethyl-1,3-dihydroinden-2-yl)-1H-imidazole, 5-(2-ethyl-1,3-dihydroinden-2-yl)-1H-imidazole; hydrochloride, Idazoxan, Yohimbine and pharmaceutically acceptable acid or salt thereof.

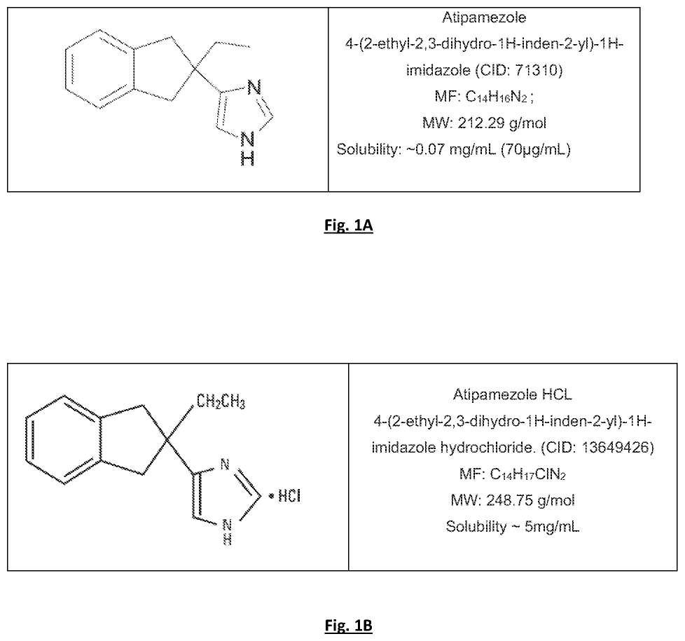

FIG. 1A depicts atipamezole in accordance with an illustrative embodiment. Atipamezole is a synthetic product highly specific to adrenergic alpha-2 receptor and acts as an antagonist. For this disclosure, the term “atipamezole” is generally used to describe either atipamezole or its acid form, atipamezole-hydrogen chloride (HCl). It has been demonstrated in multiple animal species and mammal to reverse sedation effects of alpha-2 agonists including clonidine, medetomidine, dexmedetomidine, and xylazine.

As shown in Table 1, atipamezole is reported to be one of the most potent and alpha-2 selective reversal pharmacological agent for adrenergic receptor. (based on affinity to alpha-1 and alpha-2 adrenoceptors in brain membrane receptor binding or displacement of substrate).

TABLE 1
Alpha-1, [³H]prazosin displacement; Alpha-2, [³H]clonidine
displacement. [Adapted from Pertovaara A et al, 2005, CNS
Drug Review Vol11, No 3, pp 273-288] (Pertovaara et al. 2006)

| | Alpha-2 | Alpha-1 | Alpha-2toAlpha-2 |
|---|---|---|---|
| Compound | Ki (nM) | Ki (nM) | ratio (specificity) |
| Atipamezole | 1.6 | 13,300 | 8,300 |
| Idazoxan | 148 | 3,960 | 27 |
| Yohimbine | 130 | 5,130 | 40 |

In another aspect, the key attributes considered for using atipamezole to reverse the over-sedation effects of alpha-2 agonists such as xylazine and dexmedetomidine are summarized below in Table 2.

TABLE 2
Key Attributes Considered for Using Atipamezole to
Reverse Oversedation Effects of Alpha-2 Agonists [Adapted
from Pertovaara A et al, 2005, CNS Drug Review Vol11,
No 3, pp273-288](Pertovaara et al. 2006).

High affinity and specificity to competitively inhibit alpha-2 sedative such
as xylazine and the like.
No effect on other adrenergic, opioid or GABA (Gamma-AminoButyric
Acid class) sedative and analgesic agents.
Negligible or no effect on other neurotransmitter receptors including I₂
and 5-HT (5-HydroxyTryptamine).
At 30-100 mg/dose in human with no major untoward or Severe Adverse
Event (SAE) other than increase in heart rate and blood pressure by about
14-17 mmHg (which may benefit those who are under deep respiratory
depression and breathing rate under drug overdose conditions).
Rapid breathing rate reversal of xylazine sedative effects within a few
minutes using 10-30 mg/dose; and slowly allows one to recover from
sedation.
Short elimination half-life (~2 h in humans) improving safety
Bioavailability of intramuscular, oral and buccal (similar to nasal mucosa
absorption characteristic) are known.
Reversibility and dose-effect against alpha-2 sedative agent has been
defined in humans.

In certain aspects, due to recent illicit opioid fentanyl laced with alpha-2 agonist xylazine, it is important to consider potential interactions of reversal agents or two antidotes that will work in reversing both opioid and alpha-2 agonist. In this aspect, atipamezole has been tested in combination with opioid antidote, naltrexone. In this example, a prospective study was done in white-tailed deer. Having both naltrexone and low-dose atipamezole enable the white-tailed deer to recover breathing rate notable with initial head up before other signs of recovery. In addition, the same dose given via intranasal route also provide simpler dosing with about 2 times slower, but still acceptable rate of breathing rate recovery.

Clonidine hydrochloride is an alpha-2-adrenergic agonist indicated in combination with opiates for the treatment of severe pain in cancer patients that is not adequately relieved by opioid analgesics alone. Epidural clonidine is more likely to be effective in patients with neuropathic pain than somatic or visceral pain, The safety of this drug product has only been established in a highly selected group of cancer patients, and only after an adequate trial of opioid analgesia. Other use is of unproven safety and is not recommended. In a rare patient, the potential benefits may outweigh the known risks.

Tizanidine tablets are a central alpha-2-adrenergic agonist indicated for the management of spasticity (a Multiple Sclerosis or MS drug). Because of the short duration of therapeutic effect, treatment with tizanidine tablets should be reserved for those daily activities and times when relief of spasticity is most important.

As indicated in Table 3 below, data collected with 2 antidote combination, opioid reversal naltrexone (1.5 mg/kg; an opioid antagonist similar to naloxone that inhibit opioid receptor) and atipamezole (0.1 mg/kg; alpha-2 antagonist intended to inhibit effects of agonist medetomidine), reversed the deep sedation due to dosing with carfentanil (15 μg/kg; an opioid similar to fentanyl) and medetomidine (20 μg/kg; an alpha-2 sedative similar to xylazine) in a comparison of intramuscular vs intranasal dosing in ˜54 kg white-tailed deer. N=7 per treatment group.

TABLE 3
Effects of intranasal versus intrmuscular dosing of atipamezole
and naltrexone to reverse an alpha-2 sedative, metomidine
and an opioid receptor agonist, carfentanil in reversing
sedations in deer. Please see the text for additional experimental
conditions. [Adapted from Shurny et al, Can Vet J 2010;
51: 501-505](Shury, Caulkett, and Woodbury 2010)

| | Recovery Time (min), mean ± SD | |
|---|---|---|
| Parameter of | Intra- | |
| Reversal (time) | muscular (IM) | Intranasal (IN) |
| Head up (breathing rate recovering) | 2.9 ± 2.3 | 6.8 ± 2.2 |
| Sternal Recumbency | 6 ± 4 | 9.1 ± 5.8 |
| Standing | 7.6 ± 4.2 | 9.5 ± 5.4 |

While atipamezole is not yet approved by the FDA as reversal agent for sedative in humans, there are human plasma drug concentrations established, initially with available and approved veterinary product of atipamezole. Table 5 shows pharmacokinetics of atipamezole dose response after a large volume (20 mL) intravenous infusion of atipamezole in a healthcare setting to establish exposure and rate of drug reversal in humans. A dose ranging, and clinical observation provide safety profile. The human data below in Table 5 clearly established that atipamezole is safe up to 100 mg dose and the product exhibit approximately 2-hour elimination half-life. In another aspect, this set of data also established that 20 mL and IV infusion for 2 minutes. This approach is not workable for onsite application by lay and health care professional outside of a hospital, clinic or ambulance. A smaller volume and easier, faster administration mode is required for use at point-of-care. In urgent and emergency situations without the presence of a health care provider, a product that is readily available, ready to use, easy to administer, fast onset at the point of care is needed.

TABLE 5
Pharmacokinetics of atipamezole dose-response after IV administration
(20 mL over 2 min). [n = 8 human volunteers]

| Atipamezole | Pharmacokinetic parameters | | | |
|---|---|---|---|---|
| (μg/kg) | AUC (ng · h/mL) | Cₘₐₓ (ng/mL) | Tₘₐₓ (h) | T_{1/2} (h) |
| 15 | 15.6 | 9.5 | 0.25 | 2.04 |
| 50 | 45.5 | 30 | 0.25 | 1.92 |
| 150 | 141.8 | 98.7 | 0.25 | 1.83 |

[adapted from Scheinin H et al., Anesthesiology 1998, 89: 574-584](Scheinin et al. 1998)

In another aspect, it will be even easier to provide care on site if a product can be formulated to be administered in more than one mode of administration. It is surprising to find that atipamezole can be delivered through mucosal membrane and available in the blood where drug sampling is done. In humans, oral mucosal and nasal mucosal are similar in drug absorption characteristics. The effects data collected in above mentioned white-tailed deer sedative reversal is consistent with human pharmacokinetic data described below in Table 6. While overcoming sedation may need higher concentrations, the concentrations of atipamezole for restoring breathing rate is around 10-20 ng/mL. From these human exposure data regarding atipamezole clearly demonstrate delivery by injection (IM/SC/IV) and buccal and nasal mucosa (intranasal (IN) or buccal) in humans. Oral dosing may not be easily administered for people under deep sedation, or the exposure (AUC pre equivalent dose) is too low to provide meaningful response. Thus, injectable by IM/SC or IN dosing is suitable for atipamezole dosing onsite for restoring respiratory rate and save lives in overdose situation.

TABLE 6
Human Pharmacokinetics data comparing the effects of route
of atipamezole (20 mg) dosing; investigated routes in
human consist of intravenous (low 4 mL volume in bolus
dose), oral and submucosal [n = 6 human volunteers]

| | Pharmacokinetic parameters | | | |
|---|---|---|---|---|
| | AUC | Cₘₐₓ | Tₘₐₓ | T_{1/2} |
| Rout of Administration | (ng · h/mL) | (ng/mL) | (h) | (h) |
| Intravenous | 231 | 145 | — | 1.8 |
| Mucosa (buccal similar to nasal) | 85 | 23 | 0.76 | 2.0 |
| PO (oral) | 4.2 | 1.2 | 0.82 | 2.0 |

[adapted from Huupponen et al., Cin Pharmacol & Ther 1995, 58: 506-511](Huupponen et al. 1995)

Unfortunately, stable injectable atipamezole hydrogen chloride (HCL) concentration has near solubility limit of 5 mg/mL. Atipamezole concentration higher than 5 mg/mL is not available or approved for veterinary use by the FDA's Center for Veterinary Medicine (CVM). The low concentration at 5 mg/mL would require 10 mL or 20 mL for a 50 mg or 100 mg human dose. This volume is not feasible or easily done by intramuscular (IM) or subcutaneous (SC) injection on site. It is even more challenging to dose this volume by nasal route. Nasal cavity capacity is 2 mL or less; 3 mL is the maximum IM injection volume and preferable volume is 2 mL. This poses challenges to produce point-of-care antidote even with highly potent alpha-2 antagonist.

FIG. 1B depicts an atipamezole-HCl structure in accordance with an illustrative embodiment. In some aspects, stable injectable atipamezole HCl concentration has near solubility limit of 5 mg/mL. Atipamezole concentration higher than 5 mg/mL is not available or approved for veterinary use by the FDA. The low concentration at 5 mg/ml would require 10 mL or 20 mL for a 50 mg or 100 mg human dose. This volume is not feasible or easily done by IM/SC injection on site since 3 mL is the maximum IM injection volume and preferable volume is 2 mL. It is even more challenging to dose this volume by nasal route since the nasal cavity capacity is 2 mL or less. This poses challenges to produce point-of-care antidote even with a highly potent alpha-2 antagonist.

In an embodiment, Table 7 shows increasing concentrations of atipamezole from 5 mg/mL up to 60 mg/mL suitable for injection and intranasal administration. When traditional solvation methods were applied to dissolve atipamezole in water, even with heating, agitation or sonication, atipamezole solution saturated at about 5-6 mg/mL. Unexpectedly, when 0.25 to 1 molar equivalent of HCl to atipamezole was used, it was found that atipamezole was solubilized at higher concentrations in aqueous and pharmaceutically acceptable alcoholic solvents, propylene glycol and ethanol. In another aspect, it was found that the sequence of addition is important, as using the acidified alcoholic-water as solvent without following the steps prescribed in Table 7 resulted in insoluble suspension. The ratio of HCl to atipamezole was about 0.25 to 1 molar equivalent or about 0.038 to 0.14 w/w HCl to atipamezole ratio.

TABLE 7
Preparation of a sterile, high atipamezole concentration composition in a pharmaceutically
acceptable formulation for injectable and nasal/mucosal dosing

Composition A.
In an embodiment, a method to prepare 10 or 20 mg/mL of atipamezole-HCl in sterile
solution comprises:
Step 1. In each mL of water for injection, add about 0.07 × 40 mg of HCl to make acidic
water.
Step 2. Add about 40 mg atipamezole mechanical agitation, sonication and warm to 45° C.
put into solution
Step 3. Add about 1 or 3 mL of water for injection (Quantum satis to about 2 mL or 4 mL
total volume to achieve target nominal concentration of about 10-20 mg/mL of
atipamezole-HCl)
Step 4. Passage through 0.2 μm filter to produce sterile solution
This composition is stable and suitable for storage at room temperatures between 23-27° C.
Composition B.
In an embodiment, a method to prepare 60 mg/mL of atipamezole-HCl for IM, IV, nasal
and buccal dosage forms comprises:
Step 1. In each mL of propylene glycol containing HCl (acid) equivalent to about 0.14 ×
120 HCl.
Step 2. Add about 120 mg atipamezole agitate and put into solution (elevate temperature to
about 50° C. as appropriate)
Step 3. Add about 1 mL of water for injection (and quantum satis to about 2 mL to achieve
target nominal concentration of about 60 mg/mL of atipamezole-HCl)
Step 4. Passage through 0.2 μm filter to produce sterile solution
Step 5. Store in a closed container
This composition is stable and suitable for storage at room temperature between 23-27° C.
Composition C.
In an embodiment, a method to prepare high 50 mg/mL of atipamezole-HCl for IM and IN
dosage forms comprises:
Step 1. In each mL of ethanol, add about 0.04 × 100 mg HCl
Step 2. Add 100 mg atipamezole, agitate and put into solution (in a closed container to
prevent evaporation)
Step 3. Add about 1 mL of water for injection to a total of about 2 mL solution (target
nominal concentration is about 50 mg/mL of atipamezole-HCl)
Step 4. Passage through 0.2 μm filter to produce sterile solution.
Step 5. Store in a closed container.
This composition is stable and suitable for storage at room temperature between 23-27° C.

While addition of co-solvents could increase solubility of alpha-2 antagonists such as atipamezole, a co-solvent may be subject to micro-phase separation during temperature fluctuation or other environmental variations in storage and use. In search for a product composition that is free of mixing two-solvent system, such as ethanol-water and propylene glycol-water, we accidently found that an injectable excipient histidine forms a stochiometric and stable physical complex with atipamezole.

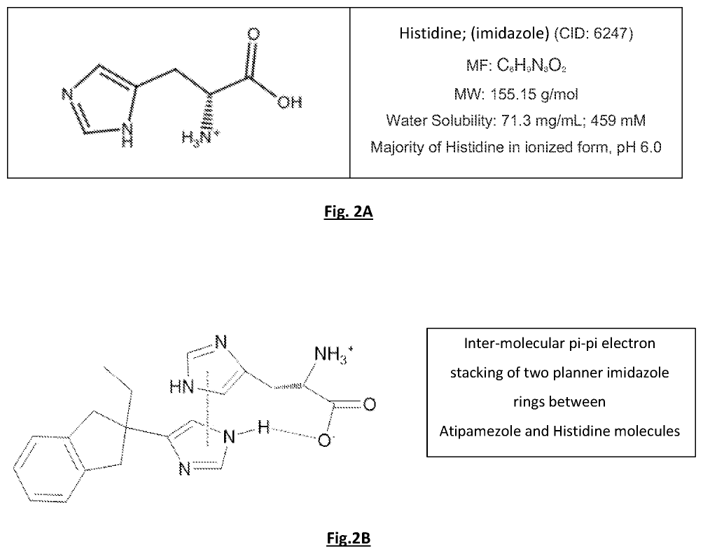

FIG. 2A depicts histidine in accordance with an illustrative embodiment. We discovered that pi-pi interactions or ring-stacking interactions and high-water solubility of histidine (with imidazole 5-member electron rich domain) can be leveraged to improve solubility of any practically insoluble drug molecules such as atipamezole with imidazole domain. Unexpectedly and under a specific process and formulation sequence, the planer imidazole of histidine could be induced to form pi-pi stacking with atipamezole. In an embodiment, practically water-insoluble atipamezole (MW=212.29 g/mol) (solubility ˜70 μg/mL) could be dissolved together with histidine (MW=155.15 g/mol) (solubility ˜71.3 mg/mL or 459.6 mM).

FIG. 2B depicts an atipamezole-histidine complex in accordance with an illustrative embodiment. In an embodiment, each mole of histidine interacts with 1 mole equivalent of atipamezole due to the non-covalent pi-stacking between the imidazole structure of atipamezole and imidazole of histidine to form a 1:1 complex in solution. Addition of excess atipamezole beyond about 459 mM and beyond that of about 97.5 mg/mL or 459.6 mM histidine leads to insoluble precipitate, and addition of more histidine did not bring about higher concentrations of atipamezole in solution). Therefore, the maximum solubility of atipamezole in atipamezole-histidine complex form is about 459 mM. With molecular weight of atipamezole of 212.29 g/mole, the final atipamezole-histidine complex in solution is 97.6 mg/mL. Thus, 80 mg/mL of atipamezole without co-solvent can be made in the presence of a pharmaceutical excipient histidine at 58.2 mg/mL.

Table 8 depicts Effects of mole-ratio between histidine and atipamezole on solubility of atipamezole in 375.8 mmol/L solution (expressed as detectable insoluble materials (denoted as +) in the suspension).

Histidine: Atipamezole

| 0 | 0.13 | 0.27 | 0.53 | 0.81 | 1.0 | 1.1 | 1.2 |
|---|---|---|---|---|---|---|---|
| * | ++++ | +++ | ++ | + | None | None | None |

*no notable atipamezole fraction in suspension.

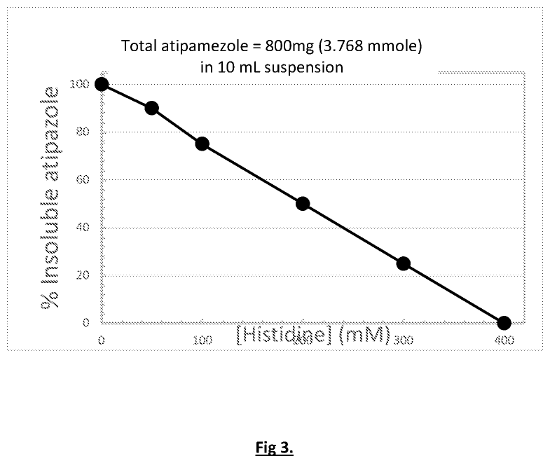

FIG. 3 depicts a chart of histidine against percentage (%) of insoluble atipamezole showing the effects of dose-response on histidine to dissolve about 800 mg or 3.768 millimole (mmol) of atipamezole in accordance with an illustrative embodiment. In the embodiment, in each vial containing 800 mg of atipamezole, 10 mL of solution containing increasing concentrations of histidine, pH of about 6 was added to resuspend atipamezole into solution at 25 degrees Celsius. The final atipamezole concentration when fully dissolved in 10 mL solution is 3.758 mmol/10 mL or 0.3758 mmol/mL which is equivalent to 375.8 millimolar (mM). This condition is recorded with 400 mM (62.1 mg/mL) histidine solution at pH 6.0. After allowing to stand for 30 min, insoluble atipamezole particles found in the suspension, detectable as turbid or cloudy insoluble fraction atipamezole were recorded accordingly. Increasing final histidine concentration did not improve or reduce atipamezole aqueous solubility.

In an embodiment, Table 9 is an exemplary preparation of a composition of atipamezole-histidine in a pharmaceutically acceptable formulation including application in vial or appropriate applicators such as syringes for nasal or injectable administering. The methods described schematically below use a scalable process using 1000 mL batch as an example to achieve an 80 mg/mL atipamezole in solution prepared with histidine serving as an excipient.

Composition D
Step 1. In 2 L container, add 80 g atipamezole powder.
Step 2. Prepare 65 mg/mL histidine at about pH 6.
Step 3. Add 900 mL of histidine to atipamezole.
Step 4. Gentle mixing at room temperature for about 30 minutes to form
the atipamezole-histidine in solution at about pH 6.0.
Step 5. Bring the volume to 1000 mL with deionized water.
To prepare for nasal or injectable dosage form
Step 1. Sterilize the solution through a 0.2 μm filter.
Step 2. Fill 1-5 mL into each injection vial.
[As an option, the atipamezole-histidine can be freeze dried or dehydrated
to provide lyophilized product for onsite hydration].
Step 3. Fill 2-3 mL syringe for ready to use administering.
Composition E
Step 1. In each 5 mL vial containing 400 mg of atipamezole powder was
prepared by ethanol solvent evaporation or lyophilization
Step 2. Add 5 mL of sterile 21.7 mg/mL histidine with pH of about 5.5 to
about 6 to the atipamezole powder in the vial.
Step 4. Passage through 0.2 μm filter to produce sterile product.
Step 5. Store in a closed container.
This composition with high atipamezole presented as atipamezole-histidine
complex, pH 5.5-6 is stable and suitable for storage at room temperature
between 23-27° C.

The following examples shown in Table 10 provide the estimated volume needed for IM and IN dosing which is within 2 mL target volume (based on current human dose range for respiratory rate reversal in overdose with alpha-2 agonist such as xylazine). One or more of the compositions described in the embodiments herein are suitable for packaging in 2 mL volume per dose in ready-to-use injector or IN applicator as described below. These devices are well-established, available from multiple vendors and many people are already familiar with its use.

TABLE 10
Volume estimate for the target dose range for human
use intended for IM, IV, nasal and buccal dosing

| | | Volume per dose | | | | |
|---|---|---|---|---|---|---|
| | | Compo- | | Compo- | Compo- | Compo- |
| Atipamezole | | sition A | | sition B | sition C | sitions D, E |
| Dose | | (20 mg/mL) | | (60 mg/mL) | (50 mg/mL) | (80 mg/mL) |
| 20 | mg | 1 | mL | 0.33 mL | 0.4 mL | 0.25 mL |
| 50 | mg | 2.5 | mL | 0.83 mL | 1.0 mL | 0.63 mL |
| 100 | mg | 5.0 | mL | 1.67 mL | 2.0 mL | 1.25 mL |

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “illustrative” is used herein to mean “serving as an example, instance or illustration.” Any embodiment or design described herein as “illustrative” is not necessarily to be construed as preferred or advantageous over other embodiments or designs. The terms “at least one” and “one or more” are understood to include any integer number greater than or equal to one, i.e., one, two, three, four, etc. The terms “a plurality” are understood to include any integer number greater than or equal to two, i.e., two, three, four, five, etc. The term “connection” can include an indirect “connection” and a direct “connection.”

References in the specification to “one embodiment,” “an embodiment,” “an example embodiment,” etc., indicate that the embodiment described can include a particular feature, structure, or characteristic, but every embodiment may or may not include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

Although the above embodiments of present invention each have been described by stating their individual advantages, respectively, present invention is not limited to a particular combination thereof. To the contrary, such embodiments may also be combined in any way and number according to the intended deployment of present invention without losing their beneficial effects.

What is claimed is:

1. A composition for reversing an alpha-2 adrenoceptor agonist in a subject comprising an alpha-2 antagonist and a histidine in a pharmaceutically acceptable formulation.

2. The composition of claim 1 wherein the composition comprises a pi-pi interaction between the alpha-2 antagonist and the histidine.

3. The composition of claim 1 wherein the alpha-2 antagonist comprises from about 80 mg/mL to 97.6 mg/mL.

4. The composition of claim 1 wherein the histidine comprises from about 300 mM to 400 mM.

5. The composition of claim 1 wherein the alpha-2 adrenoceptor agonist is selected from the group consisting of xylazine, dexmedetomidine and clonidine.

6. The composition of claim 1 wherein the alpha-2 antagonist is atipamezole.

7. The composition of claim 1 wherein the composition is administered via a route selected from the group consisting of intravenous administration, injection administration, nasal administration and buccal administration.

8. The composition of claim 1 wherein the composition has a pH of about 5.5 to about 6.

9. A composition for reversing an alpha-2 adrenoceptor agonist in a subject comprising alpha-2 antagonist and hydrogen chloride in a pharmaceutically acceptable formulation.

10. The composition of claim 9 wherein the alpha-2 antagonist comprises of atipamezole from about 20 mg/mL to 50 mg/mL.

11. The composition of claim 9 the weight ratio of hydrogen chloride to alpha-2 antagonist is from about 0.038 to 0.14.

12. The composition of claim 9 wherein the composition is administered via a route selected from the group consisting of intravenous administration, injection administration, nasal administration and buccal administration.

13. The composition of claim 9 wherein the alpha-2 adrenoceptor agonist is selected from the group consisting of xylazine, dexmedetomidine and clonidine.

14. The composition of claim 9 wherein the composition has a pH of about 5.5 to about 6.

15. A method for reversing an alpha-2 adrenoceptor agonist in a subject comprising administering a composition of alpha-2 antagonist and histidine in a pharmaceutically acceptable formulation.

16. The method for reversing an alpha-2 adrenoceptor agonist in the subject of claim 15 wherein the alpha-2 antagonist comprises atipamezole.

17. The method for reversing an alpha-2 adrenoceptor agonist in the subject of claim 15 wherein the composition is administered via a route selected from the group consisting of intravenous administration, injection administration, nasal administration and buccal administration.

18. The method for reversing an alpha-2 adrenoceptor agonist in the subject of claim 15 wherein the histidine comprises from about 300 mM to 400 mM.

19. The method for reversing an alpha-2 adrenoceptor agonist in the subject of claim 15 wherein the alpha-2 antagonist comprises from about 80 mg/mL to 97.6 mg/mL.

20. The method for reversing an alpha-2 adrenoceptor agonist in the subject of claim 15 wherein the alpha-2 adrenoceptor agonist is selected from the group consisting of xylazine, dexmedetomidine and clonidine.
